(19) United States
(12) Patent Application Publication
(10) Pub. No.: US 2006/0079510 A1
(43) Pub. Date: Apr. 13, 2006

(54) Use of PARP-1 inhibitors for protecting tumorcidal lymphocytes from apoptosis

(76) Inventors: Kristoffer Hellstrand, Goteborg (SE); Svante Hermodsson, Molndal (SE); Fredrik Thoren, Goteborg (SE); Ana Romero, Boras (SE)

Correspondence Address: KNOBBE MARTENS OLSON & BEAR LLP, 2040 MAIN STREET, FOURTEENTH FLOOR, IRVINE, CA, 92614, US

(21) Appl. No.: 11/240,014

(22) Filed: Sep. 29, 2005

Related U.S. Application Data
(60) Provisional application No. 60/614,841, filed on Sep. 30, 2004.

(51) Int. Cl. A61K 31/551; A61K 31/517; A61K 31/52; A61K 31/4745; A61K 31/4709

(52) U.S. Cl. 514/220; 514/252.17; 514/266.3; 514/310; 514/266.21; 514/417; 514/263.2; 514/300; 514/298; 514/400

(57) ABSTRACT

Method and composition for protecting tumorcidal lymphocytes including cytotoxic lymphocytes and NK cells from apoptosis and down regulation are provided. The method and composition include the administration of an effective amount of a PARP-1 inhibitor to a population of cytotoxic T lymphocytes and NK cells in the presence of monocytes or macrophages. In some embodiments, the method and composition additionally include the administration of a reactive oxygen metabolite (ROM) production or release inhibitory compound. Methods of treating cancer, viral diseases, and inflammatory diseases with a PARP-1 inhibitor are likewise provided.

### RELATED APPLICATIONS

This application claims priority under 35 U.S.C. § 119(e) to U.S. Provisional Patent Application No. 60/614,841, filed on Sep. 30, 2004, which is hereby expressly incorporated by reference in its entirety. The present application is related to U.S. patent application Ser. No. 10/680,865, filed on Oct. 7, 2003, which is a Continuation-In-Part of U.S. patent application Ser. No. 09/616,622, filed Jul. 14, 2000, now abandoned, which claims priority to U.S. Provisional Patent Application No. 60/144,394, filed on Jul. 16, 1999, all of which are hereby expressly incorporated by reference in their entireties.

### BACKGROUND OF THE INVENTION

1. Field of the Invention

The present invention relates to compositions and methods for treating cancer and/or infectious disease. More particularly, the invention provides a method for inhibiting Poly(ADP-ribose) polymerase-1 (PARP-1) dependent cell death in tumorcidal lymphocytes and NK cells.

2. Description of the Related Art

The immune system has evolved complex mechanisms for recognizing and destroying foreign cells or organisms present in the body of the host. Harnessing the body's immune mechanisms is an attractive approach to achieving effective treatment of malignancies and viral infections.

The immune system has two types of responses to foreign bodies based on the components which mediate the response: a humoral response and a cell-mediated response. The humoral response is mediated by antibodies while the cell-mediated response involves cells classified as lymphocytes. Recent anticancer and antiviral strategies have focused on utilizing the cell-mediated host immune system as a means of anticancer or antiviral treatment or therapy. A brief review of the immune system will assist in placing the teachings herein in context.

Generation of an Immune Response

The immune system functions in three phases to protect the host from foreign bodies: the cognitive phase, the activation phase, and the effector phase. In the cognitive phase, the immune system recognizes and signals the presence of a foreign antigen or invader in the body. The foreign antigen can be, for example, a cell surface marker from a neoplastic cell or a viral protein. Once the system is aware of an invading body, the cells of the immune system proliferate and differentiate in response to the invader-triggered signals. The last stage is the effector stage in which the effector cells of the immune system respond to and neutralize the detected invader.

A wide array of effector cells implement an immune response to an invader. One type of effector cell, the B cell, generates antibodies targeted against foreign antigens encountered by the host. In combination with the complement system, antibodies direct the destruction of cells or organisms bearing the targeted antigen.

Another type of effector cell is the cytotoxic lymphocyte. The natural killer cell (NK cell) is one type of cytotoxic lymphocyte, which has the capacity to spontaneously recognize and destroy a variety of virus infected cells as well as malignant cell types. The method used by NK cells to recognize target cells is poorly understood.

Another type of cytotoxic lymphocyte is the T-cell. T-cells are divided into three subcategories, each playing a different role in the immune response. Helper T-cells secrete cytokines which stimulate the proliferation of other cells necessary for mounting an effective immune response, while suppressor T-cells down regulate the immune response. A third category of T-cell, the cytotoxic T-cell (CTL), is capable of directly lysing a targeted cell presenting a foreign antigen on its surface.

The Major Histocompatability Complex and T Cell Target Recognition

T-cells are antigen specific immune cells that function in response to specific antigen signals. B lymphocytes and the antibodies they produce are also antigen-specific entities. However, unlike B lymphocytes, T-cells do not respond to antigens in a free or soluble form. For a T-cell to respond to an antigen, it requires the antigen to be bound to a presenting complex known as the major histocompatibility complex (MHC).

MHC complex proteins provide the means by which T-cells differentiate native or “self” cells from foreign cells. There are two types of MHC, class I MHC and class II MHC. T Helper cells (CD4⁺) predominately interact with class II MHC proteins while cytolytic T-cells (CD8⁺) predominately interact with class I MHC proteins. Both MHC complexes are transmembrane proteins with a majority of their structure on the external surface of the cell. Additionally, both classes of MHC have a peptide binding cleft on their external portions. It is in this cleft that small fragments of proteins, native or foreign, are bound and presented to the extracellular environment.

Cells called antigen presenting cells (APCs) display antigens to T-cells using the MHC complexes. For T-cells to recognize an antigen, it must be presented on the MHC complex for recognition. This requirement is called MHC restriction and it is the mechanism by which T-cells differentiate “self” from “non-self” cells. If an antigen is not displayed by a recognizable MHC complex, the T-cell will not recognize and act on the antigen signal.

T-cells specific for the peptide bound to a recognizable MHC complex bind to these MHC-peptide complexes and proceed to the next stage of the immune response.

Cytokines Involved in Mediating the Immune Response

The interplay between the various effector cells listed above is influenced by the activities of a wide variety of chemical factors which serve to enhance or reduce the immune response as needed. Such chemical modulators may be produced by the effector cells themselves and may influence the activity of immune cells of the same or different type as the factor producing cell.

One category of chemical mediators of the immune response is cytokines, molecules which stimulate a proliferative response in the cellular components of the immune system.

Interleukin-2 (IL-2) is a cytokine synthesized by T-cells which was first identified in conjunction with its role in the expansion of T-cells in response to an antigen (Smith, K. A. Science 240:1169 (1988)). It is well known that IL-2 secretion is necessary for the full development of cytotoxic effector T-cells (CTLs), which play an important role in the host defense against viruses. Several studies have also demonstrated that IL-2 has anti-tumor effects that make it an attractive agent for treating malignancies (see e.g. Lotze, M. T. et al, in “Interleukin 2”, ed. K. A. Smith, Academic Press, Inc., San Diego, Calif., p237 (1988); Rosenberg, S., Ann. Surgery 208:121 (1988)). In fact, IL-2 has been utilized to treat subjects suffering from malignant melanoma, renal cell carcinoma, and acute myelogenous leukemia. (Rosenberg, S. A., et al., N. Eng. J. Med. 316:889-897 (1978); Bukowski, R. M., et al., J. Clin. Oncol 7:477-485 (1989); Foa, R., et al., Br. J. Haematol. 77:491-496 (1990)).

Another cytokine with promise as an anticancer and antiviral agent is interferon-α. Interferon-α (IFN-α), an IFN type I cytokine, has been employed to treat leukemia, myeloma, and renal cell carcinomas. IFN type I cytokines have been shown to increase class I MHC molecule expression. Because most cytolytic T-cells (CTLs) recognize foreign antigens bound to class I MHC molecules, type I IFNs may boost the effector phase of cell-mediated immune responses by enhancing the efficiency of CTL-mediated killing. At the same time, type I IFN may inhibit the cognitive phase of immune responses, by preventing the activation of class II MHC-restricted helper T-cells. IL-12, IL-15, and various flavonoids can also increase the T-cell response.

In Vivo Results of Histamine Agonist Treatments

Histamine is a biogenic amine, i.e. an amino acid that possesses biological activity mediated by pharmacological receptors after decarboxylation. The role of histamine in immediate type hypersensitivity is well established. (Plaut, M. and Lichtenstein, L. M. 1982 Histamine and immune responses. In Pharmacology of Histamine Receptors, Ganellin, C. R. and M. E. Parsons eds. John Wright & Sons, Bristol pp. 392-435.)

Examinations of whether H₂-receptor agonists or antagonists can be applied to the treatment of cancer have yielded contradictory results. Some reports suggest that administration of histamine alone suppressed tumor growth in hosts having a malignancy. (Burtin, Cancer Lett. 12:195 (1981)). On the other hand, histamine has been reported to accelerate tumor growth in rodents. (Nordlund, J. J., et al., J. Invest. Dermatol 81:28 (1983)).

Similarly, contradictory results were obtained when the effects of histamine-receptor antagonists were evaluated. Some studies report that histamine-receptor antagonists suppress tumor development in rodents and humans. (Osband, M. E., et al., Lancet 1 (8221):636 (1981)). Other studies report that such treatment enhances tumor growth and may even induce tumors. (Barna, B. P., et al., Oncology 40:43 (1983)).

Synergistic Effects of a H₂-Receptor Agonist and IL-2

Despite the conflicting results when histamine is administered alone, recent reports clearly reveal that histamine acts synergistically with cytokines to augment the cytotoxicity of NK cells. For example, studies using histamine analogues suggest that histamine's synergistic effects are exerted through the H₂-receptors expressed on the cell surface of monocytes. (Hellstrand, K., et al., J. Immunol. 137:656 (1986)).

Histamine's synergistic effect when combined with cytokines appears to result from the suppression of a down regulation of cytotoxicity mediated by other cell types present along with the cytotoxic cells. In vitro studies with NK cells alone confirm that cytotoxicity is stimulated when IL-2 is administered. However, in the presence of monocytes, the IL-2 induced enhancement of cytotoxicity of NK cells is suppressed. (See, U.S. Pat. No. 5,348,739, which is incorporated herein by reference).

In the absence of monocytes, histamine had no effect or weakly suppressed NK mediated cytotoxicity. (Hellstrand, K., et al., J. Immunol. 137:656 (1986); Hellstrand, K. and Hermodsson, S., Int. Arch. Allergy Appl. Immunol. 92:379-389 (1990)). Yet, NK cells exposed to histamine and IL-2 in the presence of monocytes exhibit elevated levels of cytotoxicity relative to that obtained when NK cells are exposed only to IL-2 in the presence of monocytes. Id. Thus, the synergistic enhancement of NK cell cytotoxicity by combined histamine and interleukin-2 treatment results not from the direct action of histamine on NK cells but rather from suppression of an inhibitory signal generated by monocytes.

Granulocytes have also been shown to suppress IL-2 induced NK-cell cytotoxicity in vitro. It appears that the H₂-receptor is involved in transducing histamine's synergistic effects on overcoming granulocyte mediated suppression. For example, the effect of histamine on granulocyte mediated suppression of antibody dependent cytotoxicity of NK cells was blocked by the H₂-receptor antagonist ranitidine and mimicked by the H₂-receptor agonist dimaprit. In contrast to the complete or nearly complete abrogation of monocyte mediated NK cell suppression by histamine and IL-2, such treatment only partially removed granulocyte mediated NK cell suppression. (U.S. Pat. No. 5,348,739; Hellstrand, K., et al., Histaminergic regulation of antibody dependent cellular cytotoxicity of granulocytes, monocytes and natural killer cells., J. Leukoc. Biol 55:392-397 (1994)).

As suggested by the experiments above, therapies employing histamine and cytokines are effective anticancer and antiviral strategies. U.S. Pat. No. 5,348,739 discloses that mice given histamine and IL-2 prior to inoculation with melanoma cell lines were protected against the development of lung metastatic foci. It has also been shown that a single dose of histamine could prolong survival time in animals inoculated intravenously with herpes simplex virus (HSV), and a synergistic effect on the survival time of animals treated with a combination of histamine and IL-2 was observed (Hellstrand, K., et al., Role of histamine in natural killer cell-dependent protection against herpes simplex virus type 2 infection in mice., Clin. Diagn. Lab. Immunol. 2:277-280 (1995)).

The above results demonstrate that strategies employing a combination of histamine and IL-2 are an effective means of treating malignancies and viral infection.

Presently, the therapeutic potential of several immune cell stimulating compounds that show promise as efficacious anticancer and antiviral agents is diminished due to negatively regulating systems of the immune system. Accordingly, there is a need for methods which maximize the therapeutic potential of immune cell stimulating compounds

### SUMMARY OF THE INVENTION

The disclosed invention relates to a method of protecting cytotoxic T lymphocytes and NK cells in a subject, for the treatment of tumors, viral diseases or inflammatory diseases. Advantageously, the method includes identifying a subject in need of cytotoxic T lymphocyte and NK cell protection, administering to the subject an effective amount of a PARP-1 inhibitor effective to protect cytotoxic T lymphocytes and NK cells in the presence of monocytes or macrophages, and optionally administering an effective amount of an ROM production or release inhibitory compound.

In one aspect of the invention, the PARP-1 inhibitor can be 3-aminobenzamide; 4-amino-1,8-naphthalimide; 1,5-isoquinolinediol; 6(5H)-phenanthidone; 1,3,4,5,-tetrahydrobenzo(c)(1,6)- and (c)(1,7)-naphthyridin-6-ones; adenosine substituted 2,3-dihydro-1H-isoindol-1-ones; AG14361; 2-(4-chlorphenyl)-5-quinoxalinecarboxamide; 5-chloro-2-[3-(4-phenyl-3,6-dihydro-1 (2H)-pyridinyl)propyl]-4(3H)-quinazolinone; isoindolinone derivative INO-1001; 4-hydroxyquinazoline; 2-[3-[4-(4-chlorophenyl)-1-piperazinyl]propyl]-4-3(4)-quinazolinone; DHIQ; 3,4-dihydro-5 [4-(1-piperidinyl)(butoxy)-1(2H)-isoquinolone; CEP-6800; GB-15427; PJ34; DPQ; or imidazobenzodiazepines. Advantageously, the effective amount of the PARP-1 inhibitor is between about 10 and 500 mg/day. Alternatively, the effective amount of the PARP-1 inhibitor can be between about 100 and 250 mg/day.

In another aspect of the invention, the ROM production or release inhibitory compound can include histamine, histamine dihydrochloride, histamine phosphate, other histamine salts, histamine esters, histamine prodrugs, histamine receptor agonists, serotonin, dimaprit, clonidine, tolazoline, impromadine, 4-methylhistamine, betazole, 5HT agonists, a histamine congener, or an endogenous histamine releasing compound. Optionally, NK cell and T cell protection can be achieved by co-administering an effective amount of a cytotoxic lymphocyte stimulatory composition to the subject. The cytotoxic lymphocyte stimulatory composition can include a vaccine adjuvant, a vaccine, a peptide, a cytokine such as IL-1, IL-2, IL-12, IL-15, IFN-α, IFN-β, and IFN-γ, or a flavonoid such as flavone acetic acids and xanthenone-4-acetic acids.

In still another aspect of the invention, the cytotoxic lymphocyte stimulatory composition can be administered in a daily dose of between 1,000 and 600,000 U/kg. The effective amount of ROM production or release inhibitory compound can be between 0.05 and 50 mg per dose. Advantageously, the ROM production or release inhibitory compound is between 1 and 500 μg/kg of patient weight per dose.

In another aspect of the invention, the PARP-1 inhibitor and the ROM production or release inhibitory compound are administered separately. The administration of the PARP-1 inhibitor and the ROM production or release inhibitory compound can be performed within 24 hours. Optionally, the method can include administering an effective amount of a ROM scavenger such as catalase, glutathione peroxidase, vitamin E, vitamin A, vitamin C, SOD, SOD mimetics, or ascorbate peroxidase. The ROM scavenger can be administered in a dose of from about 0.05 to about 50 mg/day.

In yet another aspect of the invention, the method of protecting NK cells and T cells includes the administration of a chemotherapeutic agent such as an anticancer agent like cyclophosphamide, chlorambucil, melphalan, estramustine, iphosphamide, prednimustin, busulphan, tiottepa, carmustin, lomustine, methotrexate, azathioprine, mercaptopurine, thioguanine, cytarabine, fluorouracil, vinblastine, vincristine, vindesine, etoposide, teniposide, dactinomucin, doxorubin, dunorubicine, epirubicine, bleomycin, nitomycin, cisplatin, carboplatin, procarbazine, amacrine, mitoxantron, tamoxifen, nilutamid, or aminoglutemide. Advantageously, the PARP-1 inhibitor and chemotherapeutic agent are administered concomitantly. In some embodiments, the PARP-1 inhibitor, ROM production or release inhibitory compound and chemotherapeutic agent are administered concomitantly.

A composition to protect cytotoxic T lymphocytes and NK cells in a subject, for the treatment of tumors, viral diseases or inflammatory diseases is likewise provided. The composition can include an effective amount of a PARP-1 inhibitor and an effective amount of an ROM production and release inhibitory compound in a pharmaceutically acceptable carrier. The PARP-1 inhibitor can include 3-aminobenzamide; 4-amino-1,8-naphthalimide; 1,5-isoquinolinediol; 6(5H)-phenanthidone; 1,3,4,5,-tetrahydrobenzo(c)(1,6)- and (c)(1,7)-naphthyridin-6-ones; adenosine substituted 2,3-dihydro-1H-isoindol-1-ones; AG14361; 2-(4-chlorphenyl)-5-quinoxalinecarboxamide; 5-chloro-2-[3-(4-phenyl-3,6-dihydro-[(2H)-pyridinyl) propyl]-4(3H)-quinazolinone; isoindolinone derivative INO-1001; 4-hydroxyquinazoline; 2-[3-[4-(4-chlorophenyl)-1-piperazinyl]propyl]-4-3 (4)-quinazolinone; DHIQ; 3,4-dihydro-5[4-(1-piperidinyl)(butoxy)-1(2H)-isoquinolone; CEP-6800; GB-15427; PJ34; DPQ; or imidazobenzodiazepines.

In one aspect of the invention, the composition can further include a cytotoxic lymphocyte stimulatory compound such as a vaccine adjuvant, a vaccine, a peptide, a cytokine like IL-1, IL-2, IL-12, IL-15, IFN-α, IFN-β, or IFN-γ, or a flavonoid like flavone acetic acids and xanthenone-4-acetic acids. Advantageously, the ROM production and release inhibitory compound can include histamine, histamine dihydrochloride, histamine phosphate, other histamine salts, histamine esters, histamine prodrugs, histamine receptor agonists, serotonin, dimaprit, clonidine, tolazoline, impromadine, 4-methylhistamine, betazole, 5HT agonists, a histamine congener, or an endogenous histamine releasing compound. Optionally, the composition includes a chemotherapeutic agent such as cyclophosphamide, chlorambucil, melphalan, estramustine, iphosphamide, prednimustin, busulphan, tiottepa, carmustin, lomustine, methotrexate, azathioprine, mercaptopurine, thioguanine, cytarabine, fluorouracil, vinblastine, vincristine, vindesine, etoposide, teniposide, dactinomucin, doxorubin, dunorubicine, epirubicine, bleomycin, nitomycin, cisplatin, carboplatin, procarbazine, amacrine, mitoxantron, tamoxifen, nilutamid, or aminoglutemide.

### BRIEF DESCRIPTION OF THE DRAWINGS

FIG. 1 is a bar graph depicting percent apoptosis of lymphocytes incubated with autologous mononuclear phagocytes in the presence or absence of catalase, histamine or DPI.

FIG. 2 is a histogram showing caspase-3 activation of lymphocytes incubated with mononuclear phagocytes or H₂O₂ and stained with FITC-labeled caspase inhibitor, FAM-VAD.fmk.

FIG. 3 is a histogram showing caspase-3 activation of lymphocytes incubated with phagocytes or H₂O₂ in the presence or absence of PH34 or untreated control cells and stained with FITC-labeled caspase inhibitor, FAM-VAD.fmk.

FIG. 4A shows fluorescence intensity of To-Pro-3 and mitosensor monomers of lymphocytes treated with H₂O₂ alone or in the presence of PJ34 or Z-VAD.fmk at various time points. FIG. 4B shows fluorescence intensity of To-Pro-3 and mitosensor monomers of lymphocytes treated with mononuclear phagocytes alone or in the presence of PJ34 or Z-VAD.fmk as compared to untreated control cells.

FIGS. 5A and 5C are line graphs and FIGS. 5B and 5D are bar graphs depicting percent apoptosis of lymphocytes pretreated with PARP-1 inhibitors, PJ34 or Z-VAD.fmk and subjected to mononuclear phagocytes or H₂O₂ as compared to untreated control cells.

FIG. 6 is a Western blot showing nuclear AIF of H₂O₂-treated lymphocytes compared to untreated control cells.

FIG. 7 is an agarose gel showing accumulation of nuclear AIF of lymphocytes treated with H₂O₂ in the presence or absence of Z-VAD.fmk compared to untreated control cells.

### DETAILED DESCRIPTION OF THE PREFERRED EMBODIMENT

The teachings herein relate to methods of treating conditions such as cancer, viral diseases, and inflammatory diseases by administering a Poly(ADP-ribose) polymerase-1 (PARP-1)-inhibitor alone or in combination with a reactive oxygen metabolite (ROM)-inhibitory compound and/or additional therapeutic agents. An ROM inhibitory compound is any compound or composition that inhibits the production and/or release of ROM. The administration of these various agents results in the activation and protection of cytotoxic lymphocytes from the deleterious and inhibitory effects of monocytes/macrophages, the inhibition of Poly(ADP-ribose) polymerase-1 (PARP-1), and the subsequent stimulation of the anti-cancer and anti-viral properties of cytotoxic lymphocytes.

Overactivation of the nuclear enzyme poly(ADP-ribose) polymerase 1 (PARP-1) has recently been identified as an alternative route to the triggering of cell death. PARP-1 is an enzyme which functions as a DNA damage sensor and signaling molecule, binding to both single- and double-stranded DNA breaks. Upon binding to damaged DNA, PARP-1 forms homodimers and catalyzes the cleavage of NAD+. Reactive oxygen metabolites (ROMs) are potent inducers of DNA strand breakage both in vitro and in vivo. Boulares, A. H. et al. American Journal of Respiratory Cell and Molecular Biology, 28: 322-329 (2003). The resulting DNA strand breaks trigger the activation of PARP-1, the activity of which is dependent upon binding of the enzyme to the ends of the broken DNA molecules. Althaus, F. R., et al. Mol. Cell. Biochem. 193:5-11(1999). PARP-1 catalyzes the covalent attachment of long branched chains of poly(ADP-ribose) (PAR), with nicotinamide adenine dinucleotide as its substrate, to a variety of nuclear DNA-binding proteins. Such poly(ADP-ribosyl)ation contributes to various physiologic and pathophysiologic events that are associated with DNA strand breakage, including DNA replication, repair of DNA damage, gene expression, and apoptosis. See Boulares, H., et al. J. Biol. Chem. 274:22932-22940 (1999); Ding, R. et al., J. Biol. Chem. 267: 12804-12812 (1992); and Boulares, H. et al. J. Biol. Chem. 276:38185-38192 (2001).

A significant part of the dysfunction of tumor-killing lymphocytes at the site of malignant tumor growth has been attributed to inhibitory signals from tumor-infiltrating or tumor adjacent phagocytes. The phagocytes produce and secrete reactive oxygen species via a membrane NADPH oxidase. The phagocyte-derived free radicals have been shown to trigger dysfunction and apoptosis in tumoricidal or cytotoxic lymphocytes, including NK cells and cytotoxic T-cells. Cytotoxic lymphocytes are lymphocyte that possess cytotoxic capabilities such as NK-cells and cytotoxic T-cells (CTLs). The term cytotoxic lymphocytes also encompasses non-cytotoxic cells such as T-helper cells that assist in the activation of a lymphocyte with cytotoxic capabilities. The molecular events underlying phagocyte-derived lymphocyte apoptosis are not fully understood.

PARP-1 plays profound roles in diverse cellular processes including cell death, DNA repair, and gene expression, and has therefore been an interesting target for pharmacological inhibition in various diseases, such as ischemia, cancer and inflammatory pathologies. Tentori, L., et al., (2002) Pharmacological Research 45, 73-85. In cancer, the role of PARP in DNA repair has been exploited as a potential target to increase the efficacy of chemotherapy and radiotherapy. The rationale for this use is that pharmacological inhibition of PARP could incapacitate the DNA repair systems in tumor cells and thus render them sensitive to the DNA-damaging effect of chemotherapy and radiotherapy. Tentori, L., Graziani, G. (2005) Pharmacological Research 52, 25-33.

The present invention is based, in part, on the surprising and unexpected discovery that inhibitors of PARP-1 act to reduce the incidence of lymphocyte apoptosis and increase the anti-viral and anti-cancer activities of NK-cells and CTLs. That PARP inhibitors can protect lymphocytes from phagocyte-induced cell death suggests an additional role for PARP inhibitors in malignant diseases. PARP inhibitors could also protect pivotal anti-neoplastic lymphocytes and make them more responsive to immunotherapy. Suitable PARP-1 inhibitors include, without limitation, 3-aminobenzamide; 4-amino-1,8-naphthalimide; 1,5-isoquinolinediol; 6(5H)-phenanthidone; 1,3,4,5,-tetrahydrobenzo(c)(1,6)- and (c)(1,7)-naphthyridin-6-ones; adenosine substituted 2,3-dihydro-1H-isoindol-1-ones; AG14361; 2-(4-chlorphenyl)-5-quinoxalinecarboxamide; 5-chloro-2-[3-(4-phenyl-3,6-dihydro-[(2H)-pyridinyl) propyl]-4(3H)-quinazolinone; isoindolinone derivative INO-1001; 4-hydroxyquinazoline; 2-[3-[4-(4-chlorophenyl)-1-piperazinyl]propyl]-4-3(4)-quinazolinone; DHIQ; 3,4-dihydro-5[4-(1-piperidinyl)(butoxy)-1(2H)-isoquinolone; CEP-6800; GB-15427; PJ34; DPQ; and imidazobenzodiazepines.

In addition, aspects of the invention relate to the administration of a ROM inhibitory compound with a PARP-1 inhibitor. The terms “reactive oxygen metabolite inhibitors” and “ROM inhibitory compounds” have broad meanings and encompass a number of disparate compounds. NADPH inhibitors, H₂-receptor agonists, and other compounds with H₂-receptor agonist activity, suitable for use in the teachings herein, are known in the art. Examples of suitable compounds include diphenyliodonium (DPI), histamine, histamine diphosphate, histamine dihydrochloride, and compounds with a chemical structure resembling that of histamine or serotonin, yet do not negatively affect H₂-receptor activities. Suitable compounds include, but are not limited to, DPI, histamine, dimaprit, clonidine, tolazoline, impromadine, 4-methylhistamine, betazole, histamine congeners, H₂-receptor agonists, 8-OH-DPAT, ALK-3, BMY 7378, NAN 190, lisuride, d-LSD, flesoxinan, DHE, MDL 72832, 5-CT, DP-5-CT, ipsapirone, WB 4101, ergotamine, buspirone, metergoline, spiroxatrinei, PAPP, SDZ (−) 21009, and butotenine.

In some embodiments, another therapeutic agent such as a vaccine composition is likewise administered with a PARP-1 inhibitor, resulting in an increase in lymphocyte proliferation in the presence of monocytes. The addition of other agents that are cytotoxic lymphocyte activation compounds is also contemplated. Cytotoxic lymphocyte activation compounds, including those that have an immunological stimulatory character, preferably function in a synergistic fashion with a ROM inhibitory compound. Representatives of such immunological stimulatory compounds include, without limitation, cytokines, peptides, flavonoids, antigens generally, vaccines, and vaccine adjuvants. Additional classes of agents usable with the methods disclosed herein encompass chemotherapeutic and/or antiviral agents. These methods are useful for treating neoplastic as well as viral disease.

In contemplating the treatment of individuals suffering from various neoplastic and viral diseases, the teachings herein seek to stimulate and enhance cell-mediated immunity through the inhibition of PARP-1. Cell-mediated immunity (CMI) comprises the cytotoxic lymphocyte-mediated immune response to a “foreign agent.” The CMI response differs from the antibody-mediated humoral immunity in that the active agent in CMI is a cytotoxic lymphocyte rather than an antibody protein.

Cell-mediated immunity (CMI) operates with cytotoxic lymphocytes such as NK-cells and/or T-cells (CTLs) recognizing and destroying cells displaying “foreign” antigens on their surface. In the teachings herein, a foreign agent can be a neoplastic cell or a cell infected with a virus. As such, CMI functions to eliminate foreign cells from the body. For example, CMI would target cells infected with a virus, rather than to prevent the infection of the cell. Cell-mediated immunity, unlike humoral immunity which can be effective to prevent viral infection, remains the principal mechanism of defense against established viral infections. It is also pivotal in combating neoplastic disease. Therefore, the cytotoxic lymphocyte activity enhancing aspects of the teachings herein are uniquely suited to combat neoplastic and viral diseases.

As discussed above, the immune system contains a number of different cell types, each of which serve to protect the body from foreign invasion. Certain cells of the immune system produce ROM such as hydrogen peroxide, hypohalous acids, and hydroxyl radicals to achieve this goal. T-cells are considered important effector cells responsible for the anti-tumor properties of various cytokines such as IFN-α and IL-2, observed in experimental tumor models and in human neoplastic disease. (Sabzevari, H., et al., Cancer Res. 53: 4933-4937, (1993); Hakansson, A., et al., Br. J. Cancer, 74: 670-676, (1996); Wersall and Mellstedt, Med. Oncol., 12: 69-77, (1995)). The teachings herein relate, in part, to methods where compounds which inhibit PARP-1 activity are used alone or in conjunction with an ROM inhibitory compound and/or one or more T-cell activation compounds to activate or stimulate T-cells. The teachings herein, which describe the administration of at least one PARP-1 inhibitor and, optionally, an ROM inhibitory compound, T-cell activating compound, and/or anti-cancer and anti-viral compound, provide methods to treat neoplastic disorders as well as viral infections by increasing the number and specific activity of T-cells. In a preferred embodiment, the increase in the number and specific activity of T-cells is accomplished by inhibiting PARP-1, thereby reducing the damage to and down regulation of T cells and NK cells associated with apoptosis.

A number of cytotoxic lymphocyte activation compounds are known in the art to activate and stimulate cytotoxic lymphocyte activity. The dosing, routes of administration and protocols for the use and administration of these materials can be the conventional ones, well known in the art. Generally, interleukins, cytokines and flavonoids have been shown to stimulate cytotoxic lymphocyte activity. Examples of suitable compounds are selected from the group consisting of IL-1, IL-2, IL-12, IL-15, IFN-α, IFN-β, IFN-γ and flavone acetic acid, xanthenone-4-acetic acid, and analogues or derivatives thereof.

Certain vaccines and vaccine adjuvants can also be considered cytotoxic lymphocyte activating compounds. Compounds contemplated here include a number of vaccines and vaccine adjuvants that assist administered antigens to induce rapid, potent, and long-lasting cytotoxic lymphocyte-mediated immune responses, from immunized or vaccinated individuals. Illustrative vaccines include influenza vaccines, human immunodeficiency virus vaccines, Salmonella enteritidis vaccines, hepatitis B vaccines, Boretella bronchiseptica vaccines, and tuberculosis vaccines, as well as various anticancer therapeutic vaccines such as allogeneic cancer and autologous cancer vaccines which are known in the art.

One aspect of the teachings herein is directed toward the use of a variety of vaccine adjuvants. Such agents including bacillus Calmette-Guerin (BCG), pertussis toxin (PT), cholera toxin (CT), E. coli heat-labile toxin (LT), mycobacterial 71-kDa cell wall associated protein, the vaccine adjuvant oil-in-water microemulsion MF59, microparticles prepared from the biodegradable polymers poly(lactide-co-glycolides) (PLG), immune stimulating complexes (iscoms) which are 30-40 nm cage-like structures, (which consist of glycoside molecules of the adjuvant Quil A, cholesterol and phospholipids in which antigen can be integrated), as well as other suitable compounds and compositions known in the art. Such compounds can be administered in amounts sufficient to elicit an effective immune response from an immunized individual.

The teachings herein contemplate and disclose a number of different cytotoxic lymphocyte activating compounds. These compounds can be used to form cytotoxic lymphocyte activating compositions that can be administered as a step of the methods herein to achieve the activation of a patient's cytotoxic lymphocytes. The teachings herein contemplate the use of the terms “cytotoxic lymphocyte activating compound” and “cytotoxic lymphocyte activation compositions” interchangeably. The dosing, routes of administration and protocols for the use and administration of these materials can be the conventional ones, well known in the art.

A variety of ROM scavengers, including hydrogen peroxide (H₂O₂) scavengers effective to catalyze the decomposition of intercellular H₂O₂, are also known in the art. Suitable compounds include, but are not limited to, catalase, glutathione peroxidase, vitamin E, vitamin A, vitamin C, SOD, SOD mimetics, ascorbate peroxidase, and the like.

Administration of the compounds discussed above can be practiced in vitro or in vivo. When practiced in vitro, any sterile, non-toxic route of administration can be used. When practiced in vivo, administration of the compounds discussed above can be achieved advantageously by subcutaneous, intravenous, intramuscular, intraocular, oral, transmucosal, or transdermal routes, for example by injection or by means of a controlled release mechanism. Examples of controlled release mechanisms include polymers, gels, microspheres, liposomes, tablets, capsules, suppositories, pumps, syringes, ocular inserts, transdermal formulations, lotions, creams, transnasal sprays, hydrophilic gums, microcapsules, inhalants, and colloidal drug delivery systems.

The compounds are administered in a pharmaceutically acceptable form and in substantially non-toxic quantities. A variety of forms of the compounds administered are contemplated by the teachings herein. The compounds can be administered in water with or without a surfactant such as hydroxypropyl cellulose. Dispersions are also contemplated, such as those utilizing glycerol, liquid polyethylene glycols, and oils. Antimicrobial compounds can also be added to the preparations. Injectable preparations can include sterile aqueous solutions or dispersions and powders which can be diluted or suspended in a sterile environment prior to use. Carriers such as solvents or dispersion media contain water, ethanol polyols, vegetable oils and the like can also be added to the compounds provided herein. Coatings such as lecithins and surfactants can be used to maintain the proper fluidity of the composition. Isotonic agents, such as sugars or sodium chloride, can be added, as well as products intended to delay absorption of the active compounds such as aluminum monostearate and gelatin. Sterile injectable solutions are prepared according to methods well known to those of skill in the art and can be filtered prior to storage and/or use. Sterile powders can be vacuum or freeze dried from a solution or suspension. Sustained-release preparations and formulations are also contemplated by the teachings herein. Any material used in the compositions described herein should be pharmaceutically acceptable and substantially non-toxic in the amounts employed.

Although, in some of the examples that follow the compounds are used at a single concentration, it should be understood that in the clinical setting, the compounds can be administered in multiple doses over prolonged periods of time. Typically, the compounds can be administered for periods up to about one week, and even for extended periods longer than one month or one year. In some instances, administration of the compounds can be discontinued and then resumed at a later time. A daily dose of the compounds can be administered in several doses, or it can be given as a single dose. Preferably, the amount of PARP-1 inhibitor administered is between about 10-500 mg/day. However, in each case, the dose depends on the activity of the administered compound. Appropriate doses for any particular host can be readily determined by empirical techniques well known to those of ordinary skill in the art.

In addition, the compounds can be administered separately or as a single composition (combined). If administered separately, the compounds should be given in a temporally proximate manner such that the activation of cytotoxic lymphocytes by the cytokine or other compound is enhanced. More particularly, the compounds can be given within one to twenty-four hours of each other. The administration can be by either local or by systemic injection or infusion. Other methods of administration can also be suitable.

The teachings herein also contemplate combinations of at least one PARP-1 inhibitor with cytotoxic lymphocyte activation compounds, and/or an ROM production or release inhibiting compounds and ROM scavenging compounds, anticancer compounds, and combinations of antiviral compounds. The doses, routes of administration, and protocols for the use and administration of these materials can be the conventional ones, well known in the art. For example, in one embodiment, IL-2 and IL-12 are combined with a PARP-1 inhibitor to activate a population of cytotoxic lymphocytes. In an alternative embodiment, a vaccine or an adjuvant in concert with a PARP-1 inhibitor could be used to activate a population of T-cells. In another embodiment, a PARP-1 inhibitor is combined with histamine to inhibit the production or release of ROM from monocytes during a treatment regime. Combinations of other compounds, including ROM scavengers such as catalase, glutathione peroxidase, vitamin E, vitamin A, vitamin C, SOD, SOD mimetics, and ascorbate peroxidase, for example, are also contemplated. The teachings herein further contemplate using combinations of all of the various compounds discussed above to stimulate cytotoxic lymphocytes against neoplastic and/or viral disease.

All compound preparations are provided in dosage unit forms for uniform dosage and ease of administration. Each dosage unit form contains a predetermined quantity of active ingredient calculated to produce a desired effect in association with an amount of pharmaceutically acceptable carrier. Such a dosage would therefore define an effective amount of a particular compound.

A preferred compound dosage range can be determined using techniques known to those having ordinary skill in the art. IL-2, IL-12 or IL-15 can be administered in an amount of from about 1,000 to about 600,000 U/kg/day (18 MIU/m²/day or 1 mg/m²/day); more preferably, the amount is from about 3,000 to about 200,000 U/kg/day, and even more preferably, the amount is from about 5,000 to about 10,000 U/kg/day.

IFN-α, IFN-β, and IFN-γ can also be administered in an amount of from about 1,000 to about 600,000 U/kg/day; more preferably, the amount is from about 3,000 to about 200,000 U/kg/day, and even more preferably, the amount is from about 10,000 to about 100,000 U/kg/day.

Flavonoid compounds can be administered in an amount of from about 1 to about 100,000 mg/day; more preferable, the amount is from about 5 to about 10,000 mg/day, and even more preferably, the amount is from about 50 to about 1,000 mg/day.

Commonly used doses for the compounds described herein fall within the ranges listed herein. For example, IL-2 is commonly used alone in doses of about 300,000 U/kg/day. IFN-α is commonly used at 45,000 U/kg/day. IL-12 has been used in clinical trials at doses of 0.5-1.5 μg/kg/day. Motzer, et al., Clin. Cancer Res. 4(5):1183-1191 (1998). IL-1 beta has been used at 0.005 to 0.2 μg/kg/day in cancer patients. Triozzi, et al., J. Clin. Oncol. 13(2):482-489 (1995). IL-15 has been used in rates in doses of 25-400 μg/kg/day. Cao, et al., Cancer Res 58(8):1695-1699 (1998).

Vaccines and vaccine adjuvants can be administered in amounts appropriate to those individual compounds to activate cytotoxic lymphocytes. Appropriate doses for each can readily be determined by techniques well known to those of ordinary skill in the art. Such a determination will be based, in part, on the tolerability and efficacy of a particular dose using techniques similar to those used to determine proper chemotherapeutic doses.

Compounds effective to inhibit the release or formation of intercellular hydrogen peroxide, or scavengers of hydrogen peroxide, can be administered in an effective amount from about 0.05 to about 10 mg/day; more preferable, the amount is from about 0.1 to about 8 mg/day, and even more preferably, the amount is from about 0.5 to about 5 mg/day. Alternatively, these compounds can be administered from 1 to 100 micrograms per kilogram of patient body weight (1 to 100 μg/kg). However, in each case, the dose depends on the activity of the administered compound. The foregoing doses are appropriate and effective for inhibitors such as DPI, histamine, H₂-receptor agonists, other intercellular ROM production or release inhibitors or ROM scavengers. Appropriate doses for any particular host can be readily determined by empirical techniques well known to those of ordinary skill in the art.

In one embodiment, the teachings herein contemplate identifying a patient in need of enhanced cytotoxic lymphocyte activity and increasing that patient's circulating blood ROM inhibitory compound concentration to an optimum, beneficial, therapeutic level so as to provide for more efficient cytotoxic lymphocyte stimulation. Such a level can be achieved through repeated injections of the compounds described herein in the course of a day, during a period of treatment.

In another embodiment, the PARP-1 inhibitor with or without an ROM inhibitory compound is administered over a treatment period of 1 to 4 weeks with injections occurring as frequently as several times daily, over a period of up to 52 weeks. In one embodiment, the PARP-1 inhibitory compound can be administered for 9 days. In still another embodiment, the PARP-1 inhibitory compound is administered for a period of 1-2 weeks, with multiple injections occurring as frequently as several times daily. This administration can be repeated every few weeks over a time period of up to 52 weeks, or longer. Additionally, the frequency of administration can be varied depending on the patient's tolerance of the treatment and the success of the treatment. For example, the administrations can occur three times per week, or even daily, for a period of up to 24 months. When an individual is administered an ROM inhibitory compound in conjunction with a PARP-1 inhibitor, the ROM inhibitory compound can likewise be administered over a treatment period of 1 to 4 weeks with injections occurring as frequently as several times daily, over a period of up to 52 weeks. In one embodiment, the PARP-1 inhibitory compound can be administered for 9 days. In still another embodiment, the PARP-1 inhibitory compound is administered for a period of 1-2 weeks, with multiple injections occurring as frequently as several times daily. This administration can be repeated every few weeks over a time period of up to 52 weeks, or longer. Additionally, the frequency of administration can be varied depending on the patient's tolerance of the treatment and the success of the treatment. For example, the administrations can occur three times per week, or even daily, for a period of up to 24 months. Preferably, the patient is administered an ROM inhibitory compound over a period of time between about one minute and thirty minutes.

Further embodiments contemplate utility with respect to the treatment of various cancers or neoplastic diseases by administering a PARP-1 inhibitor to protect lymphocytes from ROM-mediated down regulation. Malignancies against which the teachings herein can be directed include, but are not limited to, primary and metastatic malignant tumor disease, hematological malignancies such as acute and chronic myelogenous leukemia, acute and chronic lymphatic leukemia, multiple myeloma, Waldenstroms Macroglobulinemia, hairy cell leukemia, myelodysplastic syndrome, polycytaemia vera, and essential thrombocytosis. In more specific embodiments, an ROM production or release inhibitor is administered with a PARP-1 inhibitor to a subject, in order to inhibit the growth of a tumor.

The methods described herein can also be utilized alone or in combination with other anticancer therapies. When used in combination with a chemotherapeutic regime, a PARP-1 inhibitor (with or without a ROM inhibitory compound and/or a cytotoxic lymphocyte activating compound) is administered with a chemotherapeutic agent or agents. The doses, routes of administration and protocols for the use and administration of these materials can be the conventional ones, well known in the art. Representative compounds used in cancer therapy include cyclophosphamide, chlorambucil, melphalan, estramustine, iphosphamide, prednimustin, busulphan, tiottepa, carmustin, lomustine, methotrexate, azathioprine, mercaptopurine, thioguanine, cytarabine, fluorouracil, vinblastine, vincristine, vindesine, etoposide, teniposide, dactinomucin, doxorubin, dunorubicine, epirubicine, bleomycin, nitomycin, cisplatin, carboplatin, procarbazine, amacrine, mitoxantron, tamoxifen, nilutamid, and aminoglutemide. Procedures for employing these compounds against malignancies are well established. In addition, other cancer therapy compounds can also be utilized.

The teachings herein also contemplate treatment of a variety of viral diseases by administering an effective amount of a PARP-1 inhibitor. The following are merely examples of some of the viral diseases against which the teachings herein are effective. There are a number of herpetic diseases caused by herpes simplex or herpes zoster viruses including herpes facialis, herpes genitalis, herpes labialis, herpes praeputialis, herpes progenitalis, herpes menstrualis, herpetic keratitis, herpes encephalitis, herpes zoster ophthalmicus, and shingles.

In another aspect, the teachings herein are effective against viruses that cause diseases of the enteric tract, such as rotavirus-mediated disease. In still other aspects, the teachings herein are effective against various blood based infections, such as: yellow fever, dengue, ebola, Crimean-Congo hemorrhagic fever, hanta virus disease, mononucleosis, and HIV/AIDS.

Another aspect of the teachings herein is directed toward various hepatitis causing viruses. A representative group of these viruses includes: hepatitis A virus, hepatitis B virus, hepatitis C virus, hepatitis D virus, and hepatitis E virus.

In still another aspect, the teachings herein are effective against respiratory tract diseases caused by viral infections, such as: rhinovirus infection (common cold), mumps, rubella, varicella, influenza B, respiratory syncytial virus infection, measles, acute febrile pharyngitis, pharyngoconjunctival fever, and acute respiratory disease.

Another aspect of the teachings herein contemplates treatment for various cancer-linked viruses, including: adult T-cell leukemia/lymphoma (HTLVs), nasopharyngeal carcinomas, Burkitt's lymphoma (EBV), cervical carcinomas, and hepatocellular carcinomas.

In still a further aspect, the teachings herein are useful in the treatment of viral-meditated encephalitis, including: St. Louis encephalitis, Western encephalitis, and tick-borne encephalitis.

The PARP-1 inhibitor can be administered alone to treat viral infections or in combination with an ROM production or release inhibitor and/or a conventional anti-viral agent. When used in combination with an antiviral chemotherapeutic regime, a PARP-1 inhibitor with or without an ROM inhibitory compound, and optionally a cytotoxic lymphocyte activating compound are administered with an antiviral chemotherapeutic agent or agents. The doses, routes of administration and protocols for the use and administration of these materials can be the conventional ones, well known in the art. Representative compounds used in antiviral chemotherapy include idoxuridine, trifluorothymidine, adenine arabinoside, acycloguanosine, bromovinyldeoxyuridine, ribavirin, trisodium phosphophonoformate, amantadine, rimantadine, (S)-9-(2,3-Dihydroxypropyl)-adenine, 4′,6-dichloroflavan, AZT, 3′(-azido-3′-deoxythymidine), ganciclovir, didanosine (2′,3′-dideoxyinosine or ddI), zalcitabine (2′,3′-dideoxycytidine or ddC), dideoxyadenosine (ddA), nevirapine, inhibitors of the HIV protease, and other viral protease inhibitors.

The teachings herein also contemplate using a combination of anti-cancer and anti-viral agents in conjunction with the administration of a PARP-1 inhibitor.

Although not intended to be limiting, it is contemplated that the methods herein protect lymphocytes from ROM-induced down-regulation. Alternatively, cytotoxic lymphocyte protection and activation can be accomplished by altering the mechanics of antigen presentation. One theory provides that monocytes/macrophages (MO) that are also antigen presenting cells (APC) are inhibited from presenting antigens to T-cells. This inhibition might result from MO metabolic pathways dedicated to the generation of ROM that inhibit MO antigen presenting metabolic pathways, producing mutually exclusive antigen presenting or ROM producing states in MO populations. A result of the inhibition of MO antigen presentation is that T-cell populations would remain dormant in the absence of presented antigen and in the presence of ROM.

Under this theory, administration of a PARP-1 inhibitor with or without an ROM production and release inhibiting compound, such as histamine, acts to increase T-cell activity by increasing antigen presentation. Monocytes producing ROM can have a molecular switch thrown in the presence of a PARP-1 inhibitor and/or beneficial concentrations of histamine that results in a down regulation of ROM production and an increase in antigen presentation capacity. In the mutually exclusive metabolic state hypothesized above, the down regulation of ROM production results in a subsequent increase in antigen presentation pathways and thus antigen presentation. Accordingly, administration of a PARP-1 inhibitor with or without histamine or other ROM inhibiting compounds in the presence of an antigen based T-cell activator, like a vaccine, would serve to increase T-cell activity by decreasing ROM production and increasing antigen presentation.

In an alternative theory, the administration of a PARP-1 inhibitor with or without a ROM inhibitory compound, results in an increase in cytotoxic lymphocyte activity by removing ROM-induced cytotoxic lymphocyte inhibition. The inhibition of cytotoxic lymphocytes is assuaged by the administration of a PARP-1 inhibitor, which acts to reduce cellular harm associated with apoptosis including the down regulation of lymphocytes by ROS.

The examples discussed below apply the teachings herein and show that monocytes/macrophages, and particularly MO-derived reactive oxygen metabolites (ROMs), effectively suppress the activation of human cytotoxic lymphocytes even after the in vitro administration of cytotoxic lymphocyte activation compounds such IFN-α or IL-2. Furthermore, it is shown that the addition of a PARP-1 inhibitor either alone or in combination with a ROM inhibitory compound confers protection to cytotoxic lymphocytes when added to a mixture of lymphocytes and MO.

In further embodiments, the teachings herein can be used to treat inflammatory diseases. Examples of treatable inflammatory diseases include, COPD (chronic obstructive pulmonary disease), Rheumatoid Arthritis, Crohn's disease, lupus, septicaemia, meningitis, inflammatory bowel diseases and atherosclerosis, for example. Inflammatory diseases that can be treated and/or prevented with the teachings herein are disclosed in U.S. application Ser. No. 10/171,018, filed Jun. 11, 2002, to Hellstrand et al., which is expressly incorporated herein by reference in its entirety. The PARP-1 inhibitors can be used to treat and/or prevent these diseases by protecting tumorcidal lymphocytes and NK cells from apoptosis. Additionally, the ROM-inhibitors described herein, such as histamine, augment the activity of PARP-1 inhibitors in treating and/or preventing inflammatory diseases by inhibiting the release of ROM.

### EXAMPLES

Particular aspects herein can be more readily understood by reference to the following examples, which are intended to exemplify the teachings herein, without limiting their scope to the particular exemplified embodiments.

### Example 1

Subjects with AML in a first, second, subsequent or complete remission are treated in 21-day courses with IL-2 (35-50 μg (equivalent to 6.3-9×10⁵ IU) subcutaneously (s.c.). twice daily), repeated with three to six-week intermissions and continued until relapse. In cycle #1, patients receive three weeks of low dose chemotherapy consisting of 16 mg/m²/day cytarabine, and 40 mg/day thioguanine. Concomitantly, patients are injected subcutaneously with an effective amount of a pharmaceutically acceptable form of a PARP-1 inhibitor, 3-aminobenzamide. Additionally, the patients are administered an effective amount of a pharmaceutically acceptable form of histamine dihydrochloride to boost circulating histamine to a beneficial level twice daily (above 0.2 μmole/L). Histamine levels can be continually boosted to beneficial levels by administering histamine dihydrochloride by injection at 0.2 to 2.0 mg or 3-10 μg/kg twice daily in a pharmaceutically acceptable form of a ROM inhibitory compound during the IL-2 treatment. Thereafter, the subjects are allowed to rest for three to six weeks.

After the rest period at the end of the first cycle (cycle #1), the second cycle (cycle #2) is initiated. Twice daily, injections of a pharmaceutically acceptable form of 3-aminobenzamide and a ROM inhibitory compound in a sterile carrier solution are administered at 0.5 to 2.0 mg or 3-10 μg/kg subcutaneously. Cytarabine (16 mg/m²/day s.c.) and thioguanine (40 mg/day orally) are given for 21 days (or until the platelet count is ≦50×10⁹/1). In the middle week, patients receive 0.2 to 2.0 mg or 3-10 μg/kg per injection twice per day of a pharmaceutically acceptable form of histamine dihydrochloride to boost circulating histamine to beneficial levels. At the end of the three week chemotherapy treatment, patients receive 0.2 to 2.0 mg or 3-10 μg/kg per injection twice daily of a pharmaceutically acceptable form of histamine dihydrochloride and 50 mg/day of 3-aminobenzamide for a week. Thereafter, patients receive interleukin-2 for three weeks. Patients are permitted to rest for three to six weeks.

Thereafter, cycle #3 is initiated. Cycle #3 is identical to cycle #2.

Alternatively, the treatment can also include periodically boosting patient blood histamine levels by administering an effective amount of histamine dihydrochloride injected 1, 2, or more times per day over a period of one to two weeks at regular intervals, such as daily, bi-weekly, or weekly in order to achieve a beneficial blood histamine concentration. Another alternative is to provide histamine in a depot or controlled release form. A reduction in cancer is observed.

### Example 2

As detailed above, a significant part of the dysfunction of tumor-killing lymphocytes at the site of malignant tumor growth has been attributed to inhibitory signals from tumor-infiltrating or tumor-adjacent phagocytes. The phagocytes produce and secrete reactive oxygen species (“oxygen radicals”) via a membrane NADPH oxidase, and these phagocyte-derived radicals have been shown to trigger dysfunction and apoptosis in tumoricidal lymphocytes such as NK cells and cytotoxic T-cells. However, the molecular events underlying phagocyte-induced lymphocyte apoptosis are not fully understood. The role of two enzyme systems responsible for induction and execution of apoptosis, caspases and the poly(ADP-ribose) polymerase (PARP) were investigated. Human tumoricidal lymphocytes were incubated with autologous mononuclear phagocytes or with exogenously added hydrogen peroxide, and assayed for apoptotic features at various time points. Although lymphocytes that were subjected to phagocytes or exogenous hydrogen peroxide displayed apoptotic characteristics such as depolarization of the mitochondrial transmembrane potential, DNA fragmentation, binding of FITC-VAD.fmk, and Annexin V staining, they were only partially protected by the addition of the pan-caspase inhibitor Z-VAD.fmk (100 μM). In contrast, PARP-1 inhibitors, PJ34 (250 nM) or DPQ (3 μM) completely protected tumoricidal lymphocytes from phagocyte- or hydrogen peroxide-induced apoptosis and restored tumor-killing function. It is therefore believed that PARP-dependent cell death may be critically involved in phagocyte-dependent, oxygen free radical-induced cell death.

### Example 3

Subjects suffering from Hepatitis C are identified. Individuals are administered 100 mg/day of a PARP-1 inhibitor, PJ34, intravenously for a period of three weeks. A reduction in symptoms associated with Hepatitis C was observed in the treated patient populations. Subjects who received PJ34 exhibited a reduction in ROM-mediated damage and increase in cytotoxic lymphocyte activation as compared to subjects who did not receive a PARP-1 inhibitor.

### Example 4

### Combination of a PARP-1 Inhibitor and ROM Inhibitory Compound with Chemotherapeutic Agents

PARP-1 inhibitors can also be used in conjunction with ROM inhibitory compounds and chemotherapeutic agents to treat a neoplastic or viral disease. Monocyte mediated suppression can be eliminated by administration of an ROM inhibitory compound prior, during, following or throughout chemotherapy in order to facilitate activation and protection of cytotoxic lymphocytes.

Representative compounds used in cancer and antiviral therapies are described above. Other cancer and antiviral therapeutic compounds can also be utilized. Similarly, malignancies and viral diseases against which the treatment herein can be effective, and thus can be directed, are also described above. It should be noted that the amounts, routes of administration and dosage protocols for these cancer and antiviral compounds used are well known to those of skill in the art. The teachings herein are also directed toward augmenting the efficacy of these compounds, and the therapeutic results of their use. Therefore, the conventional methodologies for their use, in conjunction with the compounds and methods provided herein, are contemplated as sufficient to achieve a desired therapeutic effect.

Subjects in need of enhanced cytotoxic lymphocyte activity, because of a neoplastic disease, and/or a viral infection such as hepatitis B (HBV), hepatitis C(HCV), human immunodeficiency virus (HIV), human papilloma virus (HPV) or herpes simplex virus (HSV) type 1 or 2, or other viral infections, are administered 250 mg/day of CEP-6800, a PARP-1 inhibitor. Additionally, subjects are administered human recombinant IL-2 (Proleukin®, Eurocetus) by subcutaneous injection or by continuous infusion of 27 μg/kg/day on days 1-5 and 8-12. The subjects also receive a daily dose of 6×10⁶ U interferon-α administered by a suitable route, such as subcutaneous injection. This treatment also includes administering 0.2 to 2.0 mg or 3-10 μg/kg of histamine injected 1, 2, or more times per day in conjunction with the administration of IL-2 and/or interferon-α.

The above procedure is repeated every 4-6 weeks until an objective regression of the tumor is observed, or until improvement in the viral infection occurs. The therapy can be continued even after a first, second, or subsequent complete remission has been observed. In patients with complete responses, the therapy can be given with longer intervals between cycles.

The treatment can also include periodically boosting patient blood histamine levels by administering 0.2 to 2.0 mg or 3-10 μg/kg of histamine injected 1, 2, or more times per day over a period of one to two weeks at regular intervals, such as daily, bi-weekly, or weekly in order to establish or maintain blood histamine at a beneficial concentration, e.g., at a concentration above 0.2 μmole/L.

Additionally, the frequency of interferon-α administration can be varied depending on the patient's tolerance of the treatment and the success of the treatment. For example, interferon can be administered three times per week, or even daily, for a period of up to 24 months. Those skilled in the art are familiar varying interferon treatments to achieve both beneficial results and patient comfort. A reduction in viral infection or tumor mass is observed.

### Example 5

As described above, the methods herein can be used to enhance the activation and protection of cytotoxic lymphocyte populations using various cytotoxic lymphocyte activation compounds that result in cytotoxic lymphocyte stimulation and/or activation. Examples of ROM inhibitory compounds include, without limitation, NADPH inhibitors, H₂-receptor agonists, and H₂O₂ scavengers and inhibitors. To demonstrate the activation and protection characteristics of these compounds, lymphocytes (including NK-cells and T-cells) and monocytes were isolated from donated blood and examined for the activation characteristics when exposed various cytotoxic lymphocyte activating compounds, such as IL-2 and/or IFN-α, vaccines, vaccine adjuvants or other immunological stimulator compounds, various ROM inhibitory compounds, such as DPI (Sigma Chemicals, St. Loius, Mo.), histamine, and various H₂O₂ scavengers, such as catalase (Boehringer-Mannheim, Germany).

Peripheral venous blood was obtained as freshly prepared leukopacks from healthy blood donors at the Blood Centre, Sahlgren's Hospital, Göteborg, Sweden, to study the activation characteristics of cytotoxic lymphocytes in the presence and absence of MO, and ROM inhibitors. The blood (65 ml) was mixed with 92.5 ml Iscove's medium, 35 ml 6% Dextran (Kabi Pharmacia, Stockholm, Sweden) and 7.5 ml acid citrate dextrose (ACD) (Baxter, Deerfield, Ill.). After incubation for 15 minutes at room temperature, the supernatant was carefully layered onto Ficoll-Hypaque (Lymphoprep, Myegaard, Norway). Mononuclear cells (MNC) were collected at the interface after centrifugation at 380 g for 15 minutes at room temperature, washed twice in PBS and resuspended in Iscove's medium supplemented with 10% human AB⁺ serum. During all further separation of cells, the cell suspensions were kept in siliconized test tubes (Vacuette, Greiner, Stockholm).

The MNC were further separated into lymphocyte and monocyte (MO) populations using the counter-current centrifugal elutriation (CCE) technique originally described by Yasaka and co-workers (Yasaka, T. et al., J. Immunol., 127:1515) with modifications as described in Hansson, M., et al. (J. Immunol., 156: 42 (1996); hereby incorporated by reference). Briefly, the sedimentation rate of cells in a spinning rotor was balanced by a counter-directed flow through the chamber. By slowly increasing the flow rate, fractions of cells of well-defined sizes were collected. The MNC were resuspended in elutration buffer containing 0.5% BSA (ICN Biomedicals Inc., Aurora, Ohio) and 0.1% EDTA (VWR, Göteborg, Sweden) in buffered NaCl and fed into a Beckman J2-21 ultracentrifuge with a JE-6B rotor (Bechman Coulter Inc., Fullerton, Calif.) at 2100 rpm. A fraction with >90% MO was obtained at a flow rate of 19 ml/min. A lymphocyte fraction enriched for NK-cells (CD3⁻/56⁺ phenotype) and T-cells (CD3⁺/56⁻) was recovered at flow rates of 14-15 ml/min. This fraction contained <3% MO and consisted of CD3ε⁻/56⁺ NK-cells (45-50%), CD3ε⁺/56⁻ T-cells (35-40%), CD3ε⁻/56⁻ cells (5-10%), and CD3ε⁺/56⁺ cells (1-5%), as judged by flow cytometry. In some experiments, dynabeads (Dynal A/S, Oslo, Norway) coated with anti-CD56 were used to obtain purified lymphocyte preparations of T-cells, as described in detail by Hansson, M., et al., incorporated above.

Following fractionation, the lymphocyte mixture of T-cells and NK cells was exposed to the various experimental conditions described below and assayed for activation using the appearance of certain cell surface proteins as indicia of activation.

Lymphocytes are identifiable by certain proteins which reside on the cell surface. Different cell surface proteins reside on different classes of lymphocytes and lymphocytes in different stages of activation. These proteins have been grouped into CD classes or “clusters of differentiation” and can serve as markers for different types of cells. Labeled antibodies, specific for different cell surface proteins, that bind to the different CD markers can be used to identify the different types of T-cells and their respective states of activation.

CD3, CD4, CD8, CD69 and CD56 (an NK-cell marker) were used to identify the cytotoxic lymphocytes of interest. The CD3 group of antibodies is specific for a marker expressed on all peripheral T-cells. The CD4 group of antibodies is specific for a marker on class II MHC-restricted T-cells, also known as T helper cells. The CD8 group of antibodies recognize a marker on class I MHC-restricted T-cells, also known as CTLs or cytolytic T-cells. The CD69 group of antibodies recognizes activated T-cells and other activated immune cells. Finally, the CD56 group recognizes a heterodimer on the surface of NK-cells.

Flow cytometry was used to identify the various sub-populations of T-cells. Flow cytometry permits an investigator to examine a population of cells using a number of labeled probes to differentiate sub-populations within the larger whole. In these experiments, the CD3 marker was used to identify the sub-population of T-cells and the CD4 and CD8 markers were used to further identify the sub-population of T-cells into T helper cells and CTLs. The effects of MO exposure in the presence and absence of histamine and T-cell activation compounds were determined using the CD69 T-cell activation marker. The expression of the different markers was estimated in a lymphocyte gate using flow cytometry (as described in Hellstrand, K., et al. Cell. Immunol. 138: 44-54 (1991), and hereby incorporated by reference).

### Example 6

### ROS Released from Mononuclear Phagocytes or Exogenous Hydrogen Peroxide Induce Cell Death in Peripheral Blood Lymphocytes

After overnight incubation with mononuclear phagocytes or hydrogen peroxide (VWR, Göteborg, Sweeden), end-stage oxidant-induced cell death in lymphocytes was assayed using flow cytometry, based on the altered characteristics displayed by end-stage apoptotic cells, i.e. reduced forward scatter and increased right angle scatter.

In accordance with earlier studies, mononuclear phagocytes induced cell death in peripheral blood lymphocytes after overnight incubation. Hansson, M., et al. (1996) J Immunol 156, 42-7 and Thoren, F., et al., (2004) J Leukoc Biol 76, 1180-6. This process was mimicked by exogenously added hydrogen peroxide and was most likely mediated by reactive oxygen species (ROS) derived from the phagocytic NADPH oxidase as lymphocytes were protected from phagocyte-induced cell death by antioxidative substances, such as catalase, histamine and DPI.

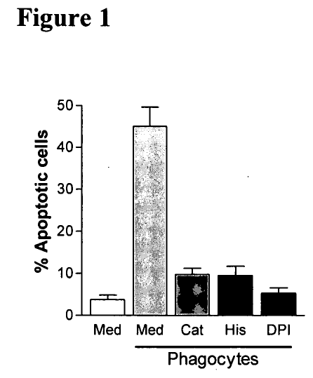

Briefly, lymphocytes were incubated overnight with autologous mononuclear phagocytes in the presence or absence of catalase (200 U/ml), histamine (1001M) or DPI (3 μM). As illustrated in FIG. 1, phagocytes induced cell death in lymphocytes (p<0.001). This effect was clearly mediated by ROS as lymphocytes were fully protected by catalase, histamine and DPI (for all, p<0.001, n=4).

### Example 7

### Caspase-3 Activation

Cell death has traditionally been divided into two forms: active programmed cell death, apoptosis, mediated by the caspase cascade which orchestrates the degradation of the cell without release of toxic substances into the surrounding tissue, and passive accidental cell death, necrosis, in which cells rapidly lose plasma membrane integrity and are degraded in an uncontrolled way. In recent years, it has become evident that apoptosis and necrosis are not always distinguishable, as dying cells can meet criteria for apoptosis and necrosis at the same time. Furthermore, recent data show that there are styles of programmed cell death in which caspases are of minor or even no importance. Lockshin, R. A., Zakeri, Z. (2002) Curr Opin Cell Biol 14, 727-33 and Jaattela, M., Tschopp, J. (2003) Nat Immunol 4, 416-23. Cell death in neural tissue commonly follows caspase-independent routes (Roy, M., Sapolsky, R. (1999) Trends Neurosci 22, 419-22 and Cregan, S. P., et al., (2002) J Cell Biol 158, 507-17), and several studies have suggested caspase-independent cell death in lymphocytes. Deas, O., et al., (1998) J Immunol 161, 3375-83, Uzzo, R. G., et al., (2001) Biochem Biophys Res Commun 287, 895-9 and Pettersen, R. D., et al., (2001) J Immunol 166, 4931-42.

To study the role of caspases for ROS-induced cell death in anti-neoplastic cells, lymphocytes subjected to phagocytes or hydrogen peroxide were assayed for binding of a fluorochrome-conjugated caspase-3 inhibitor. Briefly, lymphocytes were incubated overnight with hydrogen peroxide (250 μM) or ROS-producing phagocytes (Ph, ratio 1:1) and then assayed for caspase activation using a Fluorochrome-Labeled Inhibitor of Caspases (FLICA) assay. Lymphocytes were incubated with a FITC-labeled caspase inhibitor (FAM-VAD.fmk, MP Biomedicals, Irvine, Calif.) for one hour according to the instructions provided by the manufacturer, and the percentage of cells with active caspase-3 was determined using flow cytometry. Caspase-3 activation was also monitored using the fluorogenic caspase-3 substrate PhiPhiLux (Oncolmmunin, Gaithersburg, Md.) according to the manufacturer's instructions. The FLICA reagent traverses the membranes of all cells and bind to the active site of activated caspase 3. Thus, only cells with activated caspases will retain the reagent and become fluorescent.

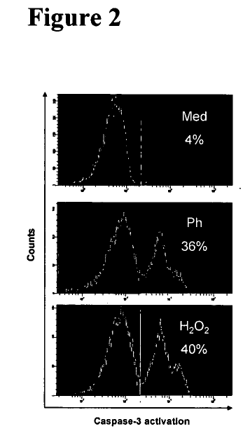

As shown in FIG. 2, lymphocytes fatally exposed to oxygen radicals bound the fluorochrome-conjugated caspase-3 inhibitor, suggesting that caspase-3 became activated during phagocyte-induced cell death.

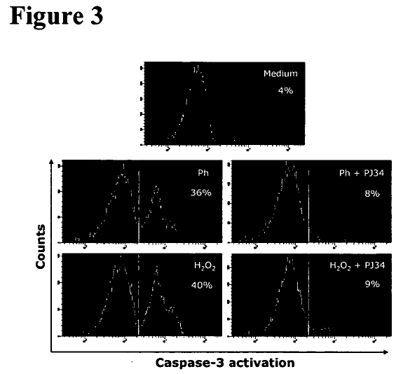

However, despite caspase activation, pre-treatment with pan-caspase inhibitors, such as Z-VAD.fmk (Sigma Chemicals, St. Louis, Mo.) and Q-VD.OPh (EMD Biosciences, La Jolla, Calif.), failed to protect lymphocytes from oxidant-induced cell death. Briefly, after overnight incubation with mononuclear phagocytes (Ph, ratio 1:1) or hydrogen peroxide (250 μM) in the presence or absence of PJ34 (250 μM), lymphocytes were stained with a FITC-labeled caspase inhibitor (FAM-VAD.fmk) and assayed for caspase activation using flow cytometry. As shown in FIG. 3, phagocytes and H₂O₂ triggered caspase activation in overnight-incubated lymphocytes. This event was reversed by pretreatment of the lymphocytes with PJ34 (250 ηM).

The failure of pan-caspase inhibitors to protect ROS-exposed lymphocytes led to an investigation as to when caspase activation occurs during the apoptotic process. Lymphocytes were subjected to phagocytes or H₂O₂ and assayed for caspase activation at different time points. It was determined that caspase activation was a rather late event in the apoptotic process (data not shown).

### Example 8

### Altered Mitochondrial Transmembrane Potential

Next, the intracellular events leading to oxidant-induced cell death were investigated. Briefly, lymphocytes were exposed to mononuclear phagocytes or hydrogen peroxide and assayed for various events associated with apoptosis. Two common events in apoptotic processes are 1) depolarization of the inner mitochondrial membrane (ΔΨₘ) and 2) exposure of phosphatidyl serine on the outside of the plasma membrane.

Depolarization of the Inner Mitochondrial Membrane (ΔΨₘ)

A Mitochondrial Membrane Sensor Kit (BD Clontech) was used to identify cells with altered mitochondrial transmembrane potential according to the manufacturer's protocol. Lymphocytes with altered Ψₘ displayed an increase in green fluorescence and a slight decrease in orange fluorescence, which could be detected using flow cytometry.

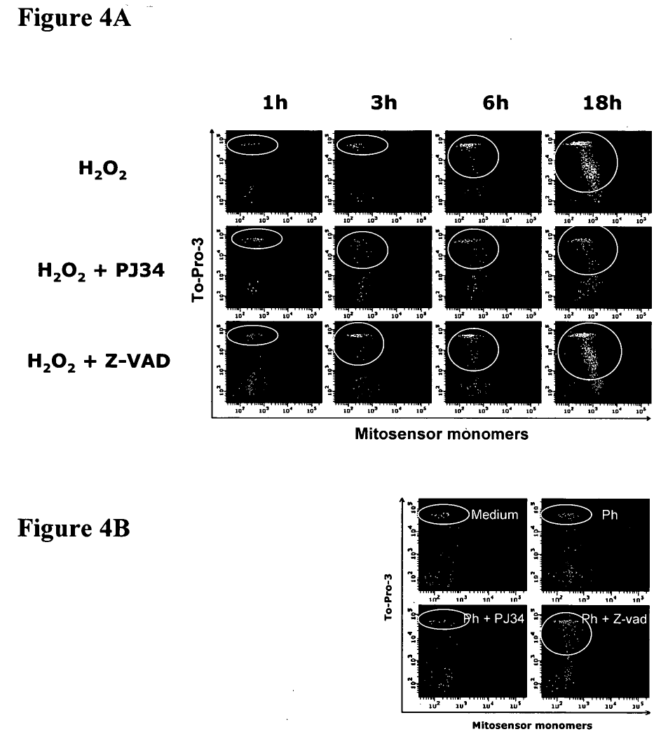

Briefly, lymphocytes exposed to hydrogen peroxide (250 μM) were assayed for altered mitochondrial transmembrane potential and plasma membrane integrity at various time points. The results are shown in FIG. 4A. Depolarization of the Ψₘ is seen as an increase in green fluorescence (mitosensor monomers). As shown in FIG. 4A, lymphocytes treated with H₂O₂ started displaying altered Ψₘ after 1 hour, and with time, more cells became apoptotic and eventually lost the integrity of the plasma membrane, as manifested by an increased To-Pro-3 (Molecular Probes) staining. PJ34 (250 μM) protected lymphocytes from oxidant-induced alterations of Ψₘ, while Z-VAD.fmk failed to display any significant protective effect against H₂O₂ or phagocytes. As shown in FIG. 4B, lymphocytes incubated with mononuclear phagocytes started displaying signs of altered Ψₘ after three hours.

Extracellular Exposure of Phosphatidyl Serine

FITC- or PE-labeled Annexin V (BD Pharmingen, San Diego, Calif.) was used to identify lymphocytes that had lost the asymmetrical distribution of membrane phospholipids and thus were exposing phosphatidyl serine on the extracellular side of the plasma membrane. Loss of structural integrity of the plasma membrane was monitored by adding the cationic dye, To-Pro-3 (1 μM) (Molecular Probes) right before the flow cytometry analysis.

Externalization of phosphatidyl serine to the outer leaflet of the plasma membrane was a later event than ΔΨₘ and was evident first after 6 hours of incubation (data not shown).

### Example 9

During the last decade, numerous reports have identified the nuclear enzyme PARP-1 as a key mediator of cell death in neural tissue after ischemia-reperfusion injury and glutamate excitotoxicity. Extensive PARP-1 activation transmits a death signal to mitochondria. The nature of this signal is not known in detail, but as a result, depolarization of the mitochondrial transmembrane potential occurs, leading to opening of high-conductance permeability pores and release of the mitochondrial protein apoptosis-inducing factor (AIF) into the cytoplasm. Thus, PARP-1-mediated cell death is accompanied with a perturbation of mitochondria, resulting in the release of AIF into the cytosol. AIF is translocated to the nucleus, where it induces DNA fragmentation. Yu, S. W., et al., (2002) Science 297, 259-63. PARP-1 activity is instrumental in various models of neural cell death, and accordingly, genetic knock-out of the gene encoding PARP-1 or pharmacological inhibition of PARP-1 elicits neuroprotection in neural models. Eliasson, M. J., et al., (1997) Nat Med 3, 1089-95, Mandir, A. S., et al., (2000) J Neurosci 20, 8005-11, and Yu, S. W., et al., (2003) Neurobiol Dis 14, 303-17

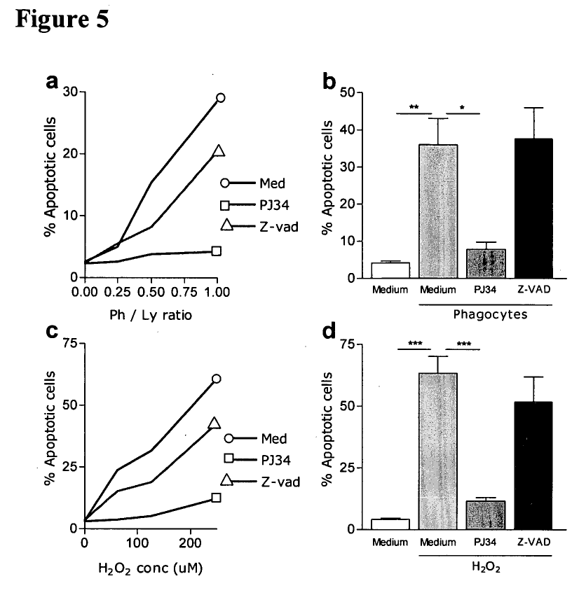

To investigate whether the PARP-AIF axis was of importance in oxidant-induced cell death in lymphocytes, lymphocytes were treated with the PARP-1 inhibitors, PJ34 and DPQ, before exposing them to phagocytes or H₂O₂. As shown in FIGS. 5A-5D, lymphocytes, pre-treated with PARP-1-inhibitors, resisted the oxidative stress imposed by phagocytes or exogenously added hydrogen peroxide.

Briefly, lymphocytes, pretreated with PJ34 (250 ηM), Z-VAD.fmk (100 μM) or medium, were subjected to mononuclear phagocytes at different Mo/Ly ratios (FIG. 5A) or to different concentrations of H₂O₂ (FIG. 5C). FIGS. 5A-5D illustrate that PJ34 protected lymphocytes from cell death induced by phagocytes (FIGS. 3A and 3B, ratio 1:1, p<0.05) and hydrogen peroxide (FIGS. 5C and 5D, 250 μM, p<0.001). Z-VAD.fmk failed to protect lymphocytes from ROS-induced cell death.

Inhibition of PARP-1 prevented ROS-induced events, such as depolarization of the inner mitochondrial membrane, exposure of phosphatidyl serine on the extracellular side of the plasma membrane, and caspase activation, the latter suggesting that caspase activation occurred down-stream of PARP-1 activation.

Immunoblotting

Nuclear extracts from lymphocytes were prepared using a NE-PER kit (Pierce) according to the instructions provided by the manufacturer. After SDS Page and western blotting, blots were incubated with a polyclonal rabbit anti-AIF antibody (Santa Cruz Biotechnology) and a HRP-conjugated goat anti-rabbit antibody (Dako) at optimized dilutions.

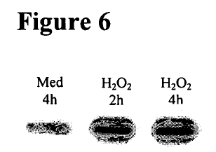

AIF was identified as the down-stream executioner of PARP-1-dependent cell-death in an in vitro-model of excitotoxic neuronal death. Yu, S. W., et al., (2002) Science 297, 259-63. Upon extensive PARP-1 activation, AIF is released from mitochondria and translocated to the nucleus (Id.), where it causes large-scale DNA fragmentation. Susin, S. A., et al., (1999) Nature 397, 441-6. To investigate the potential role of AIF in phagocyte-induced lymphocyte cell death, lymphocytes exposed to H₂O₂ were harvested at different time points and assayed for nuclear AIF by use of Western blot. As shown in FIG. 6, nuclear extracts from H₂O₂-treated lymphocytes displayed elevated levels of AIF compared to untreated control cells.

Pulsed-Field Gel Electrophoresis

Translocation of AIF to the nucleus has been associated with large-scale DNA fragmentation. The observed accumulation of nuclear AIF after challenge with H₂O₂ led to investigation as to whether large-scale DNA fragmentation accompanied lymphocyte cell death. To that end, lymphocytes were exposed to H₂O₂, cast into agarose plugs and analyzed using pulsed-field gel electrophoresis. Briefly, human lymphocytes were exposed to 250 μM H₂O₂ and incubated overnight at 37° C. After 16 hours, the cells were washed twice with PBS and resuspended in PBS. Cells were mixed with an equal volume of 2% low melting point agarose and cast into agarose plugs. After solidifying, plugs were incubated overnight at 56° C. in a buffer containing 0.2% Sodium deoxycholate and 0.5% N-lauroyl sarcosine supplemented with 0.5 mg/ml proteinase K.

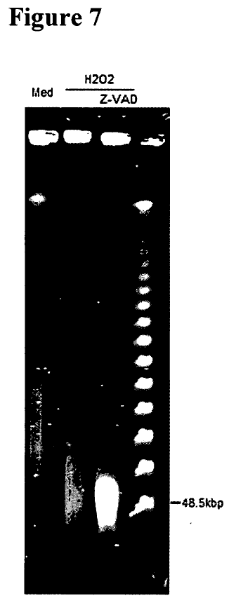

As shown in FIG. 7, a distinct band (approx. 50 kb) was seen in cells treated with Z-VAD.fmk and H₂O₂. A similar band, although less pronounced, appeared in the lane corresponding to cells treated with H₂O₂ alone. This finding suggests that large-scale chromatin fragmentation occurs in lymphocytes after ROS exposure, and that activated caspases cause partial secondary internucleosomal DNA fragmentation. However, in the presence of a pan-caspase inhibitor, the secondary fragmentation is abolished and large 50 kb-fragments accumulate.

### Discussion

The examples detailed above demonstrate that MO inhibit cytotoxic lymphocyte activation. MO inhibition of cytotoxic lymphocyte activation appears to be mediated by ROM formation. The examples also show that phagocyte-derived reactive oxygen species trigger PARP-1 and Apoptosis-Inducing Factor (AIF)-dependent cell death in human lymphocytes. The induction of cell death apparently occurs independently of caspases, as pan-caspase inhibitors failed to protect ROS-exposed lymphocytes. However, later in the apoptotic process, caspase-3 activation was observed suggesting a role for caspases in the execution phase of phagocyte-induced lymphocyte apoptosis.

The above examples show that cytotoxic lymphocytes subjected to reactive oxygen species displayed apoptotic characteristics such as depolarization of the mitochondrial transmembrane potential, caspase activation, and increased Annexin V staining. Pan-caspase inhibitors, such as Z-VAD.fmk and Q-VD-OPh, did not protect lymphocytes against oxygen radicals. In contrast, the PARP-1 inhibitors, such as PJ34 or DPQ, completely protected lymphocytes from phagocyte-derived oxygen radicals or exogenous hydrogen peroxide. The PARP-dependent cell death was accompanied by a reduction of mitochondrial transmembrane potential in lymphocytes, nuclear accumulation of AIF and large-scale DNA fragmentation. Thus, caspase activation appears to be a late event during ROS-induced lymphocyte apoptosis. In contrast, it appears that PARP/AIF axis are involved in phagocyte-dependent, oxygen radical-induced lymphocyte apoptosis.

These examples are the first to show a role for PARP activation in ROS-induced cell death in human lymphocytes. The examples demonstrate that the inhibition of PARP-1 can protect lymphocytes from ROM-induced down-regulation, thereby offering therapeutic options for the treatment of diseases and conditions characterized by ROM-mediated damage including cancer, viral infections, and inflammatory diseases. Moreover, the examples discussed above show that MO inhibition of cytotoxic lymphocyte is reversed through the addition of a ROM inhibitory compound such as histamine. These results illustrate that cytotoxic lymphocyte activation benefits from a down-regulation of MO inhibition through PARP-1 inhibition. The inhibition of MO is further augmented by the administration of an ROM production or release inhibitor in concert with a PARP-1 inhibitor.

### CONCLUSION

While particular embodiments of the teaching herein have been described in detail, it will be apparent to those of skill in the art that these embodiments are exemplary, rather than limiting. All references are hereby expressly incorporated by reference.

What is claimed is:

1. A method of protecting cytotoxic T lymphocytes and NK cells in a subject, for the treatment of tumors, viral diseases or inflammatory diseases, comprising:
identifying a subject in need of cytotoxic T lymphocyte and NK cell protection;
administering to the subject an effective amount of a PARP-1 inhibitor effective to protect cytotoxic T lymphocytes and NK cells in the presence of monocytes or macrophages; and
optionally administering an effective amount of an ROM production or release inhibitory compound.

2. The method of claim 1, wherein said PARP-1 inhibitor is selected from the group consisting of 3-aminobenzamide; 4-amino-1,8-naphthalimide; 1,5-isoquinolinediol; 6(5H)-phenanthidone; 1,3,4,5,-tetrahydrobenzo(c)(1,6)- and (c)(1,7)-naphthyridin-6-ones; adenosine substituted 2,3-dihydro-1H-isoindol-1-ones; AG14361; 2-(4-chlorphenyl)-5-quinoxalinecarboxamide; 5-chloro-2-[3-(4-phenyl-3,6-dihydro-1(2H)-pyridinyl) propyl]-4(3H)-quinazolinone; isoindolinone derivative INO-1001; 4-hydroxyquinazoline; 2-[3-[4-(4-chlorophenyl)-1-piperazinyl]propyl]-4-3(4)-quinazolinone; DHIQ; 3,4-dihydro-5 [4-(1-piperidinyl)(butoxy)-1(2H)-isoquinolone; CEP-6800; GB-15427; PJ34; DPQ; and imidazobenzodiazepines.

3. The method of claim 1, wherein said effective amount of said PARP-1 inhibitor is between about 10 and 500 mg/day.

4. The method of claim 1, wherein said effective amount of said PARP-1 inhibitor is between about 100 and 250 mg/day.

5. The method of claim 1, wherein said ROM production or release inhibitory compound is selected from the group consisting of histamine, histamine dihydrochloride, histamine phosphate, other histamine salts, histamine esters, histamine prodrugs, histamine receptor agonists, serotonin, dimaprit, clonidine, tolazoline, impromadine, 4-methylhistamine, betazole, 5HT agonists, a histamine congener, and an endogenous histamine releasing compound.

6. The method of claim 1, further comprising administering an effective amount of a cytotoxic lymphocyte stimulatory composition to the subject, wherein said cytotoxic lymphocyte stimulatory composition is selected from the group consisting of a vaccine adjuvant, a vaccine, a peptide, a cytokine, and a flavonoid.

7. The method of claim 6, wherein the composition is a cytokine selected from the group consisting of IL-1, IL-2, IL-12, IL-15, IFN-α, IFN-β, and IFNγ.

8. The method of claim 6, wherein the composition is a flavonoid selected from the group consisting of flavone acetic acids and xanthenone-4-acetic acids.

9. The method of claim 6, wherein said cytotoxic lymphocyte stimulatory composition is administered in a daily dose of between 1,000 and 600,000 U/kg.

10. The method of claim 1, wherein said effective amount of ROM production or release inhibitory compound is between 0.05 and 50 mg per dose.

11. The method of claim 10, wherein said effective amount of ROM production or release inhibitory compound is between 1 and 500 μg/kg of patient weight per dose.

12. The method of claim 1, wherein the administration of said PARP-1 inhibitor and said ROM production or release inhibitory compound is performed separately.

13. The method of claim 1, wherein the administration of said PARP-1 inhibitor and said ROM production or release inhibitory compound is performed within 24 hours.

14. The method of claim 1, further comprising administering an effective amount of a ROM scavenger.

15. The method of claim 14, wherein said ROM scavenger is selected from the group consisting of catalase, glutathione peroxidase, vitamin E, vitamin A, vitamin C, SOD, SOD mimetics, and ascorbate peroxidase.

16. The method of claim 14, wherein said ROM scavenger is administered in a dose of from about 0.05 to about 50 mg/day.

17. The method of claim 1, further comprising administering a chemotherapeutic agent.

18. The method of claim 17, wherein the chemotherapeutic agent comprises an anticancer agent selected from the group consisting of cyclophosphamide, chlorambucil, melphalan, estramustine, iphosphamide, prednimustin, busulphan, tiottepa, carmustin, lomustine, methotrexate, azathioprine, mercaptopurine, thioguanine, cytarabine, fluorouracil, vinblastine, vincristine, vindesine, etoposide, teniposide, dactinomucin, doxorubin, dunorubicine, epirubicine, bleomycin, nitomycin, cisplatin, carboplatin, procarbazine, amacrine, mitoxantron, tamoxifen, nilutamid, and aminoglutemide.

19. The method of claim 17, wherein administering said effective amount of PARP-1 inhibitor and said chemotherapeutic agent are performed concomitantly.

20. A composition to protect cytotoxic T lymphocytes and NK cells in a subject, for the treatment of tumors, viral diseases or inflammatory diseases, comprising an effective amount of a PARP-1 inhibitor and an effective amount of an ROM production and release inhibitory compound in a pharmaceutically acceptable carrier.

21. The composition of claim 20, wherein said PARP-1 inhibitor is selected from the group consisting of 3-aminobenzamide; 4-amino-1,8-naphthalimide; 1,5-isoquinolinediol; 6(5H)-phenanthidone; 1,3,4,5,-tetrahydrobenzo(c)(1,6)- and (c)(1,7)-naphthyridin-6-ones; adenosine substituted 2,3-dihydro-1H-isoindol-1-ones; AG14361; 2-(4-chlorphenyl)-5-quinoxalinecarboxamide; 5-chloro-2-[3-(4-phenyl-3,6-dihydro-1(2H)-pyridinyl) propyl]-4(3H)-quinazolinone; isoindolinone derivative INO-1001; 4-hydroxyquinazoline; 2-[3-[4-(4-chlorophenyl)-1-piperazinyl]propyl]-4-3(4)-quinazolinone; DHIQ; 3,4-dihydro-5 [4-(1-piperidinyl)(butoxy)-1(2H)-isoquinolone; CEP-6800; GB-15427; PJ34; DPQ; and imidazobenzodiazepines.

22. The composition of claim 20, further comprising a cytotoxic lymphocyte stimulatory compound selected from the group consisting of a vaccine adjuvant, a vaccine, a peptide, a cytokine, and a flavonoid.

23. The composition of claim 22, wherein the compound is a cytokine selected from the group consisting of IL-1, IL-2, IL-12, IL-15, IFN-α, IFN-β, and IFN-γ.

24. The composition of claim 22, wherein the compound is a flavonoid selected from the group consisting of flavone acetic acids and xanthenone-4-acetic acids.

25. The composition of claim 22, wherein said cytotoxic lymphocyte stimulatory composition is administered in a daily dose of between 1,000 and 600,000 U/kg.

26. The composition of claim 20, wherein said ROM production and release inhibitory compound is selected from the group consisting of histamine, histamine dihydrochloride, histamine phosphate, other histamine salts, histamine esters, histamine prodrugs, histamine receptor agonists, serotonin, dimaprit, clonidine, tolazoline, impromadine, 4-methylhistamine, betazole, 5HT agonists, a histamine congener, and an endogenous histamine releasing compound.

27. The composition of claim 20, wherein said effective amount of said ROM production or release inhibitory compound is between 0.05 and 50 mg per dose.

28. The composition of claim 20, wherein said effective amount of said ROM production or release inhibitory compound is between 1 and 500 μg/kg of patient weight per dose.

29. The composition of claim 20, further comprising a chemotherapeutic agent.

30. The composition of claim 29, wherein the chemotherapeutic agent comprises an anticancer agent selected from the group consisting of cyclophosphamide, chlorambucil, melphalan, estramustine, iphosphamide, prednimustin, busulphan, tiottepa, carmustin, lomustine, methotrexate, azathioprine, mercaptopurine, thioguanine, cytarabine, fluorouracil, vinblastine, vincristine, vindesine, etoposide, teniposide, dactinomucin, doxorubin, dunorubicine, epirubicine, bleomycin, nitomycin, cisplatin, carboplatin, procarbazine, amacrine, mitoxantron, tamoxifen, nilutamid, and aminoglutemide.

31. The composition of claim 20, wherein said effective amount of said PARP-1 inhibitor is between about 10 to about 500 mg/day.

32. The composition of claim 20, wherein said effective amount of said PARP-1 inhibitor is between about 100 and 250 mg/day.
